(19) United States
(12) Patent Application Publication
(10) Pub. No.: US 2025/0235270 A1
(43) Pub. Date: Jul. 24, 2025

(54) MARKERLESS TRACKING WITH SPECTRAL IMAGING CAMERA(S)

(71) Applicant: Monogram Orthopaedics Inc., Austin, TX (US)

(72) Inventors: Kamran Shamaei, Austin, TX (US); Pedro Alfonso Patlan Rosales, Austin, TX (US); Sanath Vurelli, Round Rock, TX (US)

(21) Appl. No.: 19/173,251

(22) Filed: Apr. 8, 2025

Related U.S. Application Data
(63) Continuation of application No. PCT/US2023/077071, filed on Oct. 17, 2023.
(60) Provisional application No. 63/379,834, filed on Oct. 17, 2022.

(51) Int. Cl. A61B 34/20; A61B 34/30

(52) U.S. Cl. CPC A61B 34/20; A61B 34/30; A61B 2034/2048; A61B 2034/2055; A61B 2034/2065

(57) ABSTRACT

Markerless tracking using spectral imaging camera(s) includes imaging, using at least one spectral imaging camera, an area that includes comprising one or more object(s), the imaging including obtaining intensity signals for a selective one or more wavelengths or wavelength ranges that correlate to selected material of at least one object of the one or more objects, using the obtained signals to determine a respective position of each of the at least one object in space, and tracking positions of the at one object in space over time.

### CROSS-REFERENCE TO RELATED APPLICATIONS

This application is a bypass continuation of International Application No. PCT/US2023/077071, filed on Oct. 17, 2023, entitled “MARKERLESS TRACKING WITH SPECTRAL IMAGING CAMERA(S)”, which international application perfects and claims priority benefit of U.S. Provisional Application No. 63/379,834, filed Oct. 17, 2023, entitled “MARKERLESS TRACKING WITH SPECTRAL IMAGING CAMERA(S)”, which applications are hereby incorporated herein by reference in their entireties.

### BACKGROUND

In various clinical applications it may be beneficial to relay to a processor information about the current and/or past locations and orientations (spatial position or pose) of an object, i.e., to track the object. Frequently, two-dimensional (2D) and three-dimensional (3D) representations of tracked objects are displayed. By way of nonlimiting example, pre-operatively captured data, such as reconstructed computed tomography (CT) views of a patient's pre-operative anatomy may be displayed by the tracking system. A tracking system that displays views of the anatomy and/or surgical instruments is sometimes referred to as a navigation system.

By way of nonlimiting example, in orthopedic joint reconstruction arthritic joints are replaced with a prosthesis. By way of nonlimiting example, in a knee replacement, a series of bone resections are made to accommodate the placement of implants.

### SUMMARY

Shortcomings of the prior art are overcome and additional advantages are provided through the provision of a computer-implemented method. The method images, using at least one spectral imaging camera, an area including one or more object(s). The imaging includes obtaining intensity signals for a selective one or more wavelengths or wavelength ranges that correlate to selected material of at least one object of the one or more objects. The method also uses the obtained signals to determine a respective position of each of the at least one object in space, and tracks positions of the at one object in space over time.

Additional aspects of the present disclosure are directed to systems and computer program products configured to perform the methods described above and herein. The present summary is not intended to illustrate each aspect of, every implementation of, and/or every embodiment of the present disclosure. Additional features and advantages are realized through the concepts described herein.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1 depicts an example of tracking anatomy via tracking arrays;

FIG. 2 depicts an example of array articulation via a series of joints;

FIG. 3 depicts an example approach for point registration;

FIG. 4 depicts an example camera with depth sensing capability;

FIG. 5 depicts an example in which cartilage obstructs the view to bone surface underneath the cartilage;

FIG. 6 depicts an example of hyperspectral imaging that assigns each pixel of a two-dimensional image a third dimension of spectral information;

FIG. 7 depicts example unique reflectance properties for each of six different materials/objects;

FIG. 8 depicts an example set of resections performed during a total knee arthroplasty;

FIG. 9 depicts anatomy of a knee joint;

FIGS. 10 & 11 depict an example exposed knee joint and anatomical features thereof; and

FIG. 12 depicts an example process for markerless tracking with spectral imaging camera(s), in accordance with aspects described herein;

FIG. 13 depicts an example computer system to perform aspects described herein.

### DETAILED DESCRIPTION

Aspects described herein present novel approaches, processes, and methods of tracking objects, for example anatomy and optionally other objects, such as surgical instruments, that does not rely on the placement of known objects into a scene. Additionally, aspects reduce surgical setup time and reduce the number of surgical steps required to conduct any navigated procedure, which can be advantageous because surgical setup time and registration time with current systems account for a significant share of the total surgical time. Additional drawbacks of current/conventional approaches exist, for instance:

Placement of rigid arrays for navigation: For navigated surgical procedures, tracking arrays are often rigidly mounted to the anatomy of interest, for example to bone. In many current approaches, the arrays are rigidly fixed with 3 millimeter (mm) bicortical bone pins that are generally 50 mm long stiffened via a sleeve between the pins. It is imperative under these approaches that the array be rigid relative to the anatomy of interest. This configuration may not provide stability of the array in all directions.

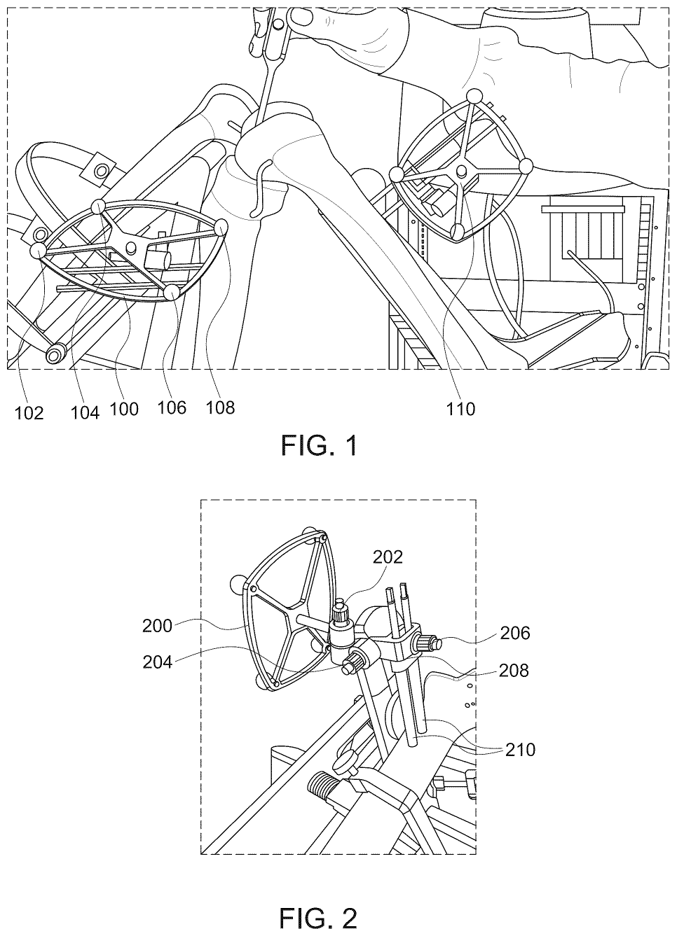

Referring to FIG. 1, to track objects of interest the localization cameras require line-of-sight to the tracker arrays 100, 110. The array fiducials (e.g., 102, 104, 106, 108 using array 100 as an example) also need to be generally oriented such that all of them are visible to the cameras. This requires full articulation of the array relative to the mount.

Conventional methods achieve this with a series of joints tightened with instrumentation. Referring to FIG. 2, articulation/movement of array 200 is provided by three points of articulation 202, 204, 206, each adjusted by loosening a bolt, making the adjustment, then tightening the bolt. Points 202 and 204 provide rotatable joints, while point 206 enables the sliding member 208 to slide along the 210 to move the array assembly nearer to, or farther from, the anatomy. In this setup, the mechanism lacks rigidity until all of the joints are tightened. This approach therefore requires some dexterity and two hands to orient and tighten, adding time, frustration and cost.

The aforementioned examples describe the placement of arrays for tracking object position optically. There may be other methods of tracking that require the placement of a fixed marker on the object of interest. By way of nonlimiting example, an approach exists that proposes rigidly placing a beacon for use of radar to track bone. In contrast, aspects described herein can eliminate the need for placement of any tracking markers, since aspects described herein provide approaches for markerless tracking, that is, tracking without use or reliance on markers. By way of nonlimiting example, placing fixed markers introduces surgical time, surgical complexity, and surgical cost. Bone pins are also invasive and are often inserted outside of the incision.

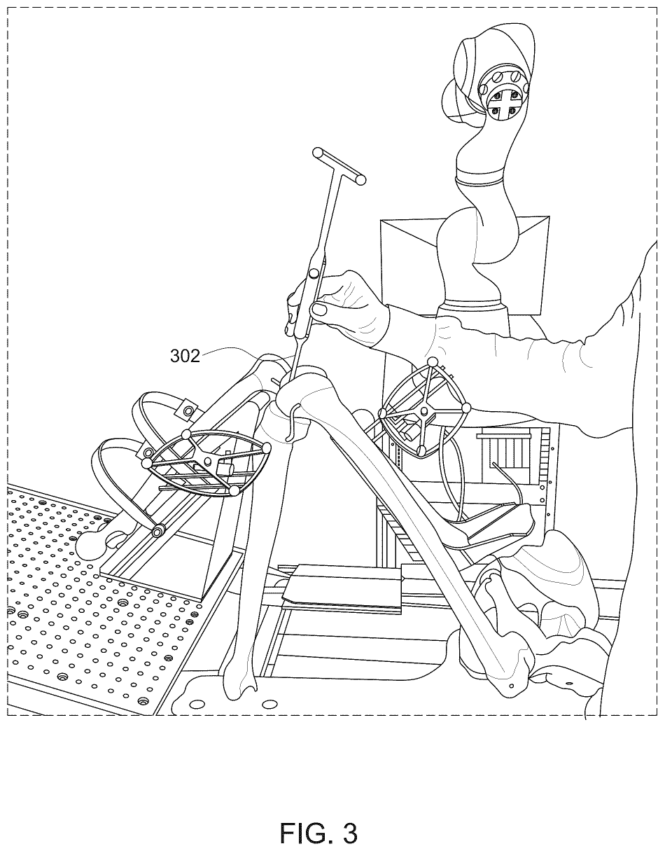

The position of the marker relative to the anatomy must be registered: Once the arrays are rigidly placed, the transform between the array position relative to the anatomy must be calculated because it is impossible to know with high accuracy where in the anatomy the marker was placed. This is generally achieved through a process known as point registration. There are several methods that could be used for this. Ultrasound is one example. Referring to FIG. 3, the most common method involves a sharp instrument 302 that probes through the cartilage in multiple places as shown in FIG. 3 to capture a point cloud of bone surface points. Various mathematical computations are then used to correlate the point cloud to pre-operative models (for example from a CT scan) or generalized anatomical models to return the object pose.

Because aspects described herein propose tracking actual object(s) and not a marker, there is no need for a user to register the position of a tracked marker relative to the anatomy of interest, i.e., the registration step is eliminated in approaches described herein.

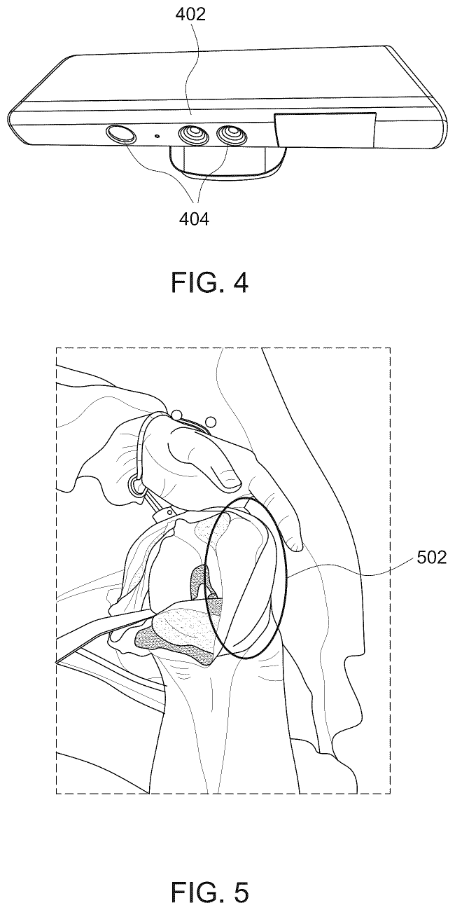

Tracking an object with a depth sensor camera instead of markers: There may be cameras that integrate, by way of nonlimiting example, a Red, Green, Blue wavelength (RGB) camera/sensor(s) and an infrared sensor. Other variations can include a structured light projector and camera receiver wherein a known pattern is projected onto a scene to calculate depth and surface information about the objects in the scene. The example camera of FIG. 4 shows an RGBD camera that incorporates an RGB camera 402 together with depth sensor(s) 404.

A limitation of such technologies is that the fast feature detection algorithms on which depth cameras rely struggle at correlating the data they are collecting to pre-operative data sets, especially when the surgical exposure is small (minimally invasive) and provides limited observable surface area and may be occluded by other objects (cartilage, blood, surgical tools and other soft tissues). But perhaps an even more significant limitation is that most pre-operative imaging is x-ray based (notably, a CT scan is a series of x-rays). Cartilage does not show up on an x-ray. X-rays are most useful for imaging bone. If a camera, such as a depth camera, is tasked with correlating a scene to pre-operative data, the scene has cartilage. Very little bone is exposed. These cameras cannot ‘see through’ the cartilage and other objects to the bone surface, which is what constitutes the pre-operative data set from the x-ray. An example of this is presented in FIG. 5, showing a surgical incision that partially exposes a bone but leaves at least a portion of the bone obstructed by cartilage in region 302.

Accordingly, aspects discussed herein address these and other drawbacks. For example:

Markerless tracking before altering the anatomy: In accordance with some aspects, spectral imaging (by way of a spectral imaging camera) is used to track objects of interest. Example spectral imaging that could be used include hyperspectral imaging (using one or more hyperspectral camera(s)) and multispectral imaging (using one or more multispectral imaging camera(s)). Hyperspectral imaging, like other spectral imaging, collects and processes information from across the electromagnetic spectrum. The goal of such imaging is to obtain spectra for each pixel in an image, with the intent of finding objects, identifying materials, or detecting processes. Whereas the human eye sees color of only the visible light spectrum in mostly three bands—long wavelengths (red), medium wavelengths (green), and short wavelengths (blue), hyperspectral imaging sees a broader range of wavelengths extending beyond those that are visible. Certain objects leave unique ‘fingerprints’ in the electromagnetic spectrum. Known as spectral signatures, these ‘fingerprints’ enable identification of the materials that make up a scanned object. By way of nonlimiting example, a parameter may be the relative absorbance of light at t wavelengths.

In other words, a hyperspectral camera is able to spatially scan, or detect, various materials irrespective of relative obstruction. In simplified terms, all objects emit radiation or absorb radiation at wavelengths that are unique to the material (like a physical material property). Hyperspectral cameras can detect this radiation within some distance of the object. For example, cartilage appears to reflect/absorb radiation at around the wavelengths of 500-600 nanometer (nm), which is sufficient for tissue identification. With the naked eye, all that is seen is cartilage, however a hyperspectral imaging camera is able to detect bone surface and other anatomy that is obstructed by the cartilage. Thus, with a hyperspectral camera, the bone surface can be ‘seen’ for purposes of determining the position of the bone surface despite the fact it is obstructed by cartilage. The bone surface is invisible to a traditional camera or depth sensor camera. Given that the pre-operative and clinical anatomy of interest is the bone surface, imaging the bone surface directly facilitates markerless tracking.

Because different organs feature unique spectral fingerprints, machine learning could be used to help identify tissues. Specifically, training datasets that provide the reflection and absorption of various wavelengths in specific materials can be used to train a neural network and/or other artificial intelligence model(s). The AI model (e.g., neural network) could then be used to identify tissues in a more generalized way.

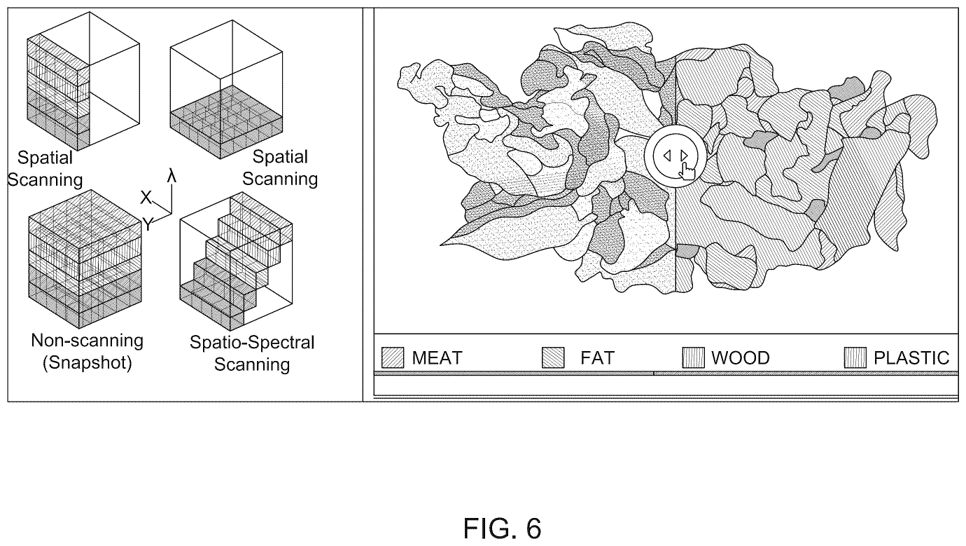

Referring to FIG. 6, hyperspectral imaging (HSI) works by assigning each pixel of a conventional two-dimensional digital image a third dimension of spectral information. The spectral information contains the wavelength-specific reflectance intensity of the pixel. This results in a three-dimensional datacube with two spatial dimensions (x, y) and a third (spectral) dimension (λ).

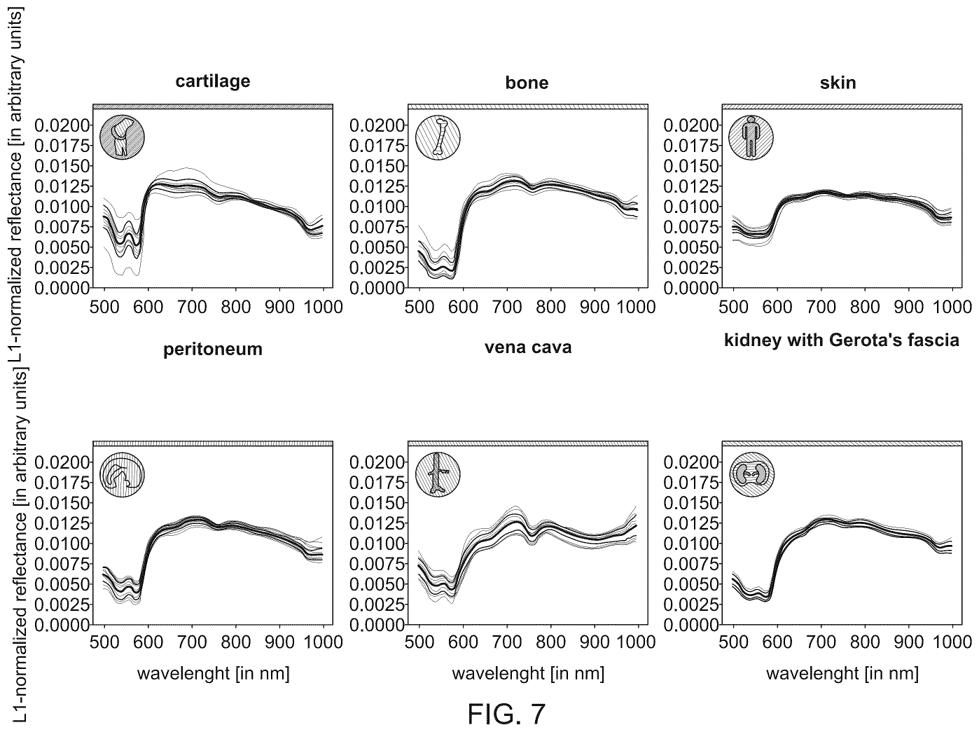

As noted, different materials have different electromagnetic signatures or properties, that is—they emit, reflect, and absorb wavelengths from a light source differently. These differences can be used to identify tissues based on their spectral signature. FIG. 7 presents example such unique reflectance properties for each of 6 different materials/objects.

To track an object of interest, a process can look for/detect an object of interest (for example, the bone) in the scene based on its electromagnetic signature and use mathematical algorithms to correlate the observed object in the scene to the preoperative dataset and return the pose (i.e., track the bone). The hyperspectral camera need not track at the region of the surgical incision. For example, it may prove more beneficial to track extra incisional regions, such as the shin versus the proximal tibia. This may be needed, if, for example, it is difficult to detect the radiation emitted or absorbed by an object because of the material that is occluding it, for example, detecting bone through cartilage.

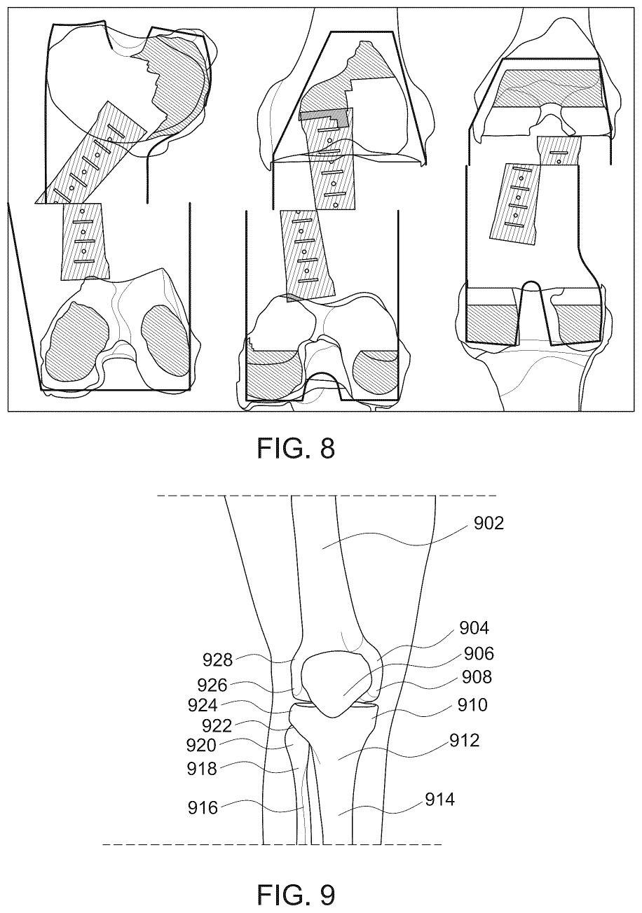

Markerless tracking after altering the anatomy (especially robotics or navigated surgical execution): By way of nonlimiting generalization, the purpose of many surgical procedures is to alter the preoperative anatomy. Navigated surgical procedures can help surgeons plan and execute such alterations to the anatomy. For example, in a robotic total knee arthroplasty, a surgeon might execute a series of resections (exhibited in FIG. 8 with six example resections) to facilitate placement of implant(s). Once the anatomy has been surgically altered, a navigation system is to keep track of any such alterations. The navigation system may need to accordingly update the pre-operative dataset to account for such alterations so that it can correlate the surgically altered anatomy as observed from the hyperspectral imaging camera to the corresponding pre-operative dataset.

The hyperspectral camera may be able to sufficiently see through skin and other anatomy to unaltered regions of bone. Thus, the hyperspectral cameras may not be required to observe regions at or around the surgical site. For example, with the tibia it may track the shin instead of the proximal tibia, as noted above.

Of particular importance may be that, if correlating to a preoperative model, the navigation system accounts for changes to the anatomy and updates the model to which it is correlating based on those changes.

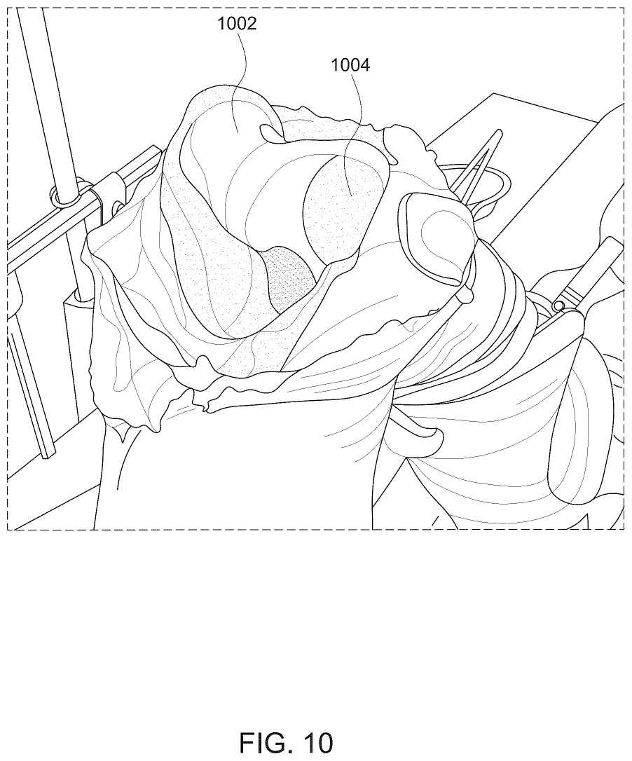

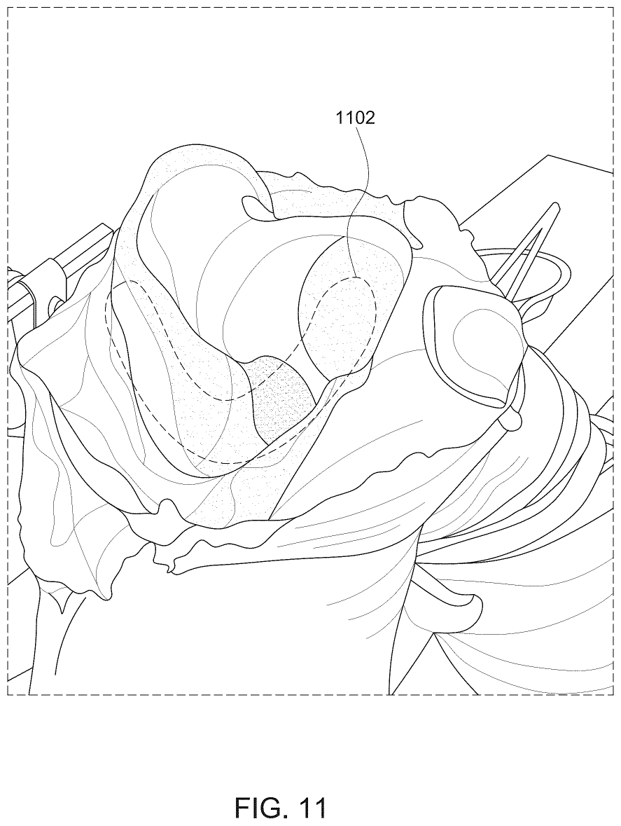

Markerless tracking for visualization of anatomy unobstructed by cartilage but that may be obstructed by other materials, for example soft tissues, ligaments, tendons, blood, fat, etc.: Certain hyperspectral cameras may be limited in their capacity to observe bone surfaces that are obstructed by cartilage. In certain embodiments, the camera(s) may be positioned such that anatomy that is not obstructed by cartilage is in the camera(s) field of view. By way of nonlimiting example, consider a knee joint of FIG. 9 with anatomy that includes the femur 902, medial femoral epicondyle 904, patella 906, medial femoral condyle 908, medial tibial condyle 910, tibial tuberosity 912, tibia 914, fibula 916, fibular neck 918, fibular head 920, proximal tibiofibular joint 922, lateral tibial condyle 924, lateral femoral condyle 926, and lateral femoral epicondyle 928. The hyperspectral camera(s) for the femur (902) may be positioned such that one or more of the following anatomical structures are in view: lateral femoral condyle 926, lateral tibial condyle 924, medial femoral epicondyle 904, medial femoral condyle 908 or any non-distal portion of the femur 902. Multiple cameras may be used to image different anatomical regions, even for the same bone. By way of nonlimiting example, the hyperspectral camera(s) for the tibia 914 may be positioned such that one or more of the following anatomical structures are in view: the medial tibial condyle 910, the tibial tuberosity 912, the shin (front face of the tibia), the lateral tibial condyle 924 or non-proximal portions of the tibia 914.

For hyperspectral cameras that do not have properties such that they can visualize a bone surface through cartilage, or if they do have such properties but for whatever reason it may be less preferable to view through the cartilage, an embodiment can visualize the bone surface in specific regions that are not obstructed by cartilage but may be obstructed by other anatomy.

Referring to FIG. 10, the hyperspectral camera may not be able to see the bone surface through cartilage 1002, or where it may be less desired to visualize through the cartilage 1002, but it may, by way of nonlimiting example, be able to see the bone surface through soft tissue of the lateral condyle/epicondyle 1004.

Referring to FIG. 11, the region shown by 1102 represents an exposed bone surface not covered by cartilage, and could be tracked by a hyperspectral camera.

Aspects described herein may be helpful for any navigated surgical procedure and other applications.

In particular examples, a hyperspectral/multispectral imaging approach is integrated into an orthopedic surgical robot, specifically as the localizing camera. The hyperspectral camera could be mounted to a cart, tripod, fixture attached to the surgical table, or to the robot, as examples. In certain embodiments, there could be multiple such cameras in various configurations in an operating room. A function of the camera(s) can be to track objects of clinical interest. In one embodiment of a system, the position of tracked objects could be used to plan surgical procedures/actions and specifically to plan the trajectories of robot mounted tools.

In general, all objects emit or absorb radiation at wavelengths that may be unique to their material properties, i.e., all materials have an electromagnetic signature. Some objects may not emit enough radiation for detection with a spectral imaging camera. To detect such objects, an external source (light at a different wavelength) may be employed such that the diffraction/absorbance of the light for different materials can be detected. In some configurations, there is a light or energy source positioned near the object of interest and illuminating the object of interest.

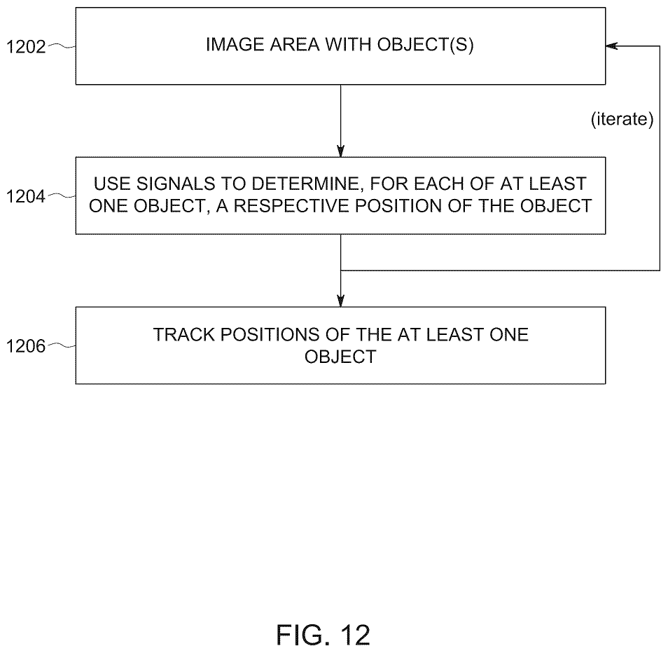

Accordingly, FIG. 12 depicts an example process for markerless tracking with spectral imaging camera(s), in accordance with aspects described herein. The process includes imaging (1202), using at least one spectral imaging camera, an area that includes one or more object(s). The imaging includes, for instance, obtaining intensity signals for a selective one or more wavelengths or wavelength ranges that correlate to selected material of at least one object of the one or more objects. The process continues by using (1204) the obtained signals to determine a respective position of each of the at least one object in space. This imaging (1202) and using (1204) can be repeated iteratively at different points in time, for instance periodically or aperiodically, to track (1206) positions of the at one object in space over time. Example spectral imaging cameras include one or more hyperspectral imaging cameras for hyperspectral imaging of the area and/or one or more multispectral imaging cameras for multispectral imaging of the area.

In examples, the area includes, at least partially, a surgical scene, and the at least one object includes patient anatomy. The patient anatomy includes, for example, bone or other selected anatomy. The process can further include correlating the determined respective position of the anatomy to a prior-obtained model of the anatomy or modified version of the prior-obtained model. The prior-obtained model can include a preoperative two-dimensional or three-dimensional model of the anatomy. In embodiments, the process tracks alterations to the anatomy during a surgical procedure and updates the prior-obtained model according to the tracked alterations to provide the modified version of the prior-obtained model, and correlates the altered anatomy as observed from the imaging to the corresponding modified version of the prior-obtained model.

The using (1204) the signals can include using at least one algorithm to correlate the anatomy to a preoperative dataset or modified version of the preoperative dataset and return a location/pose of the anatomy. In embodiments, the process tracks alterations to the anatomy during a surgical procedure and updates the preoperative dataset according to the tracked alterations to provide the modified version of the preoperative dataset, and correlating the altered anatomy as observed from the imaging to the corresponding modified version of the preoperative dataset.

Determining the position(s) may be performed absent use or reliance on tracking of fiducials or other markers on the object(s) or in the area, placement of arrays for tracking object position optically, and beacons and RADAR-based tracking.

In embodiments, the using (1204) includes applying an artificial intelligence (AI) model to identify the at least one object. The AI model may be configured to identify selected materials based on training the AI model using machine learning and at least one datasets providing reflection/absorption of various wavelengths for varying specific materials.

One or more embodiments described herein may be incorporated in, performed by, and/or used by one or more computer systems, such as one or more systems that are, or are in communication with, a camera system, tracking system, and/or orthopedic surgical robot, as examples. Processes described herein may be performed singly or collectively by one or more computer systems. A computer system may also be referred to herein as a data processing device/system, computing device/system/node, or simply a computer. The computer system may be based on one or more of various system architectures and/or instruction set architectures.

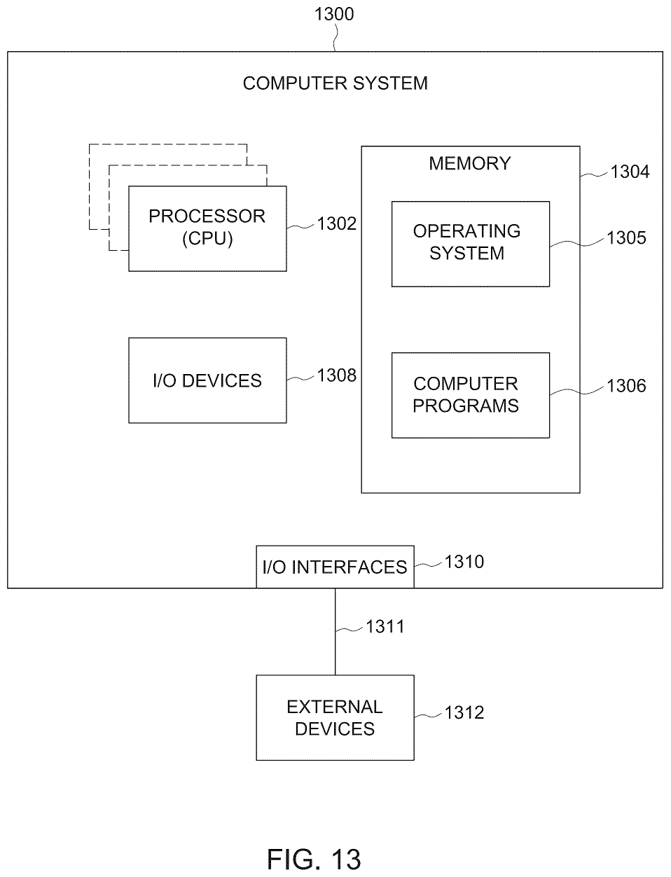

FIG. 13 shows a computer system 1300 in communication with external device(s) 1312. Computer system 1300 includes one or more processor(s) 1302, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 1302 can also include register(s) to be used by one or more of the functional components. Computer system 1300 also includes memory 1304, input/output (I/O) devices 1308, and I/O interfaces 1310, which may be coupled to processor(s) 1302 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 1304 can be or include main or system memory (e.g., Random Access Memory) used in the execution of program instructions, storage device(s) such as hard drive(s), flash media, or optical media as examples, and/or cache memory, as examples. Memory 1304 can include, for instance, a cache, such as a shared cache, which may be coupled to local caches (examples include L1 cache, L2 cache, etc.) of processor(s) 1302. Additionally, memory 1304 may be or include at least one computer program product having a set (e.g., at least one) of program modules, instructions, code or the like that is/are configured to carry out functions of embodiments described herein when executed by one or more processors.

Memory 1304 can store an operating system 1305 and other computer programs 1306, such as one or more computer programs/applications that execute to perform aspects described herein. Specifically, programs/applications can include computer readable program instructions that may be configured to carry out functions of embodiments of aspects described herein.

Examples of I/O devices 1308 include but are not limited to microphones, speakers, Global Positioning System (GPS) devices, RGB, IR, and/or spectral cameras, lights, accelerometers, gyroscopes, magnetometers, sensor devices configured to sense light, proximity, heart rate, body and/or ambient temperature, blood pressure, and/or skin resistance, registration probes and activity monitors. An I/O device may be incorporated into the computer system as shown, though in some embodiments an I/O device may be regarded as an external device (1312) coupled to the computer system through one or more I/O interfaces 1310.

Computer system 1300 may communicate with one or more external devices 1312 via one or more I/O interfaces 1310. Example external devices include a keyboard, a pointing device, a display, and/or any other devices that enable a user to interact with computer system 1300. Other example external devices include any device that enables computer system 1300 to communicate with one or more other computing systems or peripheral devices such as a printer. A network interface/adapter is an example I/O interface that enables computer system 1300 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 1310 and external devices 1312 can occur across wired and/or wireless communications link(s) 1311, such as Ethernet-based wired or wireless connections. Example wireless connections include cellular, Wi-Fi, Bluetooth®, proximity-based, near-field, or other types of wireless connections. More generally, communications link(s) 1311 may be any appropriate wireless and/or wired communication link(s) for communicating data.

Particular external device(s) 1312 may include one or more data storage devices, which may store one or more programs, one or more computer readable program instructions, and/or data, etc. Computer system 1300 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 1300 may be operational with numerous other general purpose or special purpose computing system environments or configurations. Computer system 1300 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method comprising:
imaging, using at least one spectral imaging camera, an area comprising one or more objects, wherein the imaging comprises obtaining intensity signals for a selective one or more wavelengths or wavelength ranges that correlate to selected material of at least one object of the one or more objects;
using the obtained intensity signals to determine a respective position of each object of the at least one object in space; and
tracking positions of the at one object in space over time.

2. The method of claim 1, wherein the tracking comprises repeating the imaging and the using one or more times at different points in time.

3. The method of claim 1, wherein the at least one spectral imaging camera comprises at least one selected from the group consisting of: (i) one or more hyperspectral imaging cameras for hyperspectral imaging of the area and (ii) one or more multispectral imaging cameras for multispectral imaging of the area.

4. The method of claim 1, wherein the area comprises a surgical scene and wherein the at least one object comprises patient anatomy, the patient anatomy comprising bone or other selected anatomy.

5. The method of claim 4, further comprising correlating the determined respective position of the patient anatomy to a prior-obtained model of the patient anatomy or modified version of the prior-obtained model.

6. The method of claim 5, wherein the prior-obtained model comprises a preoperative two-dimensional or three-dimensional model of the patient anatomy.

7. The method of claim 6, further comprising:
tracking alterations to the patient anatomy during a surgical procedure and updating the prior-obtained model according to the tracked alterations to provide the modified version of the prior-obtained model; and
correlating the altered patient anatomy as observed from the imaging to the modified version of the prior-obtained model.

8. The method of claim 4, wherein the using comprises using at least one algorithm to correlate the patient anatomy to a preoperative dataset or modified version of the preoperative dataset and return a location/pose of the patient anatomy.

9. The method of claim 8, further comprising:
tracking alterations to the patient anatomy during a surgical procedure and updating the preoperative dataset according to the tracked alterations to provide the modified version of the preoperative dataset; and
correlating the altered patient anatomy as observed from the imaging to the modified version of the preoperative dataset.

10. The method of claim 1, wherein the using determines the a respective position of each object of the one or more objects absent use or reliance on (i) tracking of fiducials or other markers on the one or more objects or in the area comprising the one or more objects, (ii) placement of arrays for tracking object position optically, and (iii) beacons and RADAR-based tracking.

11. The method of claim 10, wherein the using comprises applying an artificial intelligence (AI) model to identify the at least one object, the AI model configured to identify selected materials based on training the AI model using machine learning and at least one dataset providing reflection or absorption of various wavelengths for varying specific materials.

12. A computer system comprising:
a memory; and
a processing circuit in communication with the memory, wherein the computer system is configured to perform a method comprising:
imaging, using at least one spectral imaging camera, an area comprising one or more objects, wherein the imaging comprises obtaining intensity signals for a selective one or more wavelengths or wavelength ranges that correlate to selected material of at least one object of the one or more objects;
using the obtained intensity signals to determine a respective position of each object of the at least one object in space; and
tracking positions of the at one object in space over time.

13. The computer system of claim 12, wherein the tracking comprises repeating the imaging and the using one or more times at different points in time.

14. The computer system of claim 12, wherein the at least one spectral imaging camera comprises at least one selected from the group consisting of: (i) one or more hyperspectral imaging cameras for hyperspectral imaging of the area and (ii) one or more multispectral imaging cameras for multispectral imaging of the area.

15. The computer system of claim 12, wherein the area comprises a surgical scene, and wherein the at least one object comprises patient anatomy, the patient anatomy comprising bone or other selected anatomy.

16. The computer system of claim 15, wherein the method further comprises correlating the determined respective position of the patient anatomy to a prior-obtained model of the patient anatomy or modified version of the prior-obtained model.

17. The computer system of claim 16, wherein the prior-obtained model comprises a preoperative two-dimensional or three-dimensional model of the patient anatomy.

18. The method of claim 17, wherein the method further comprises:
tracking alterations to the patient anatomy during a surgical procedure and updating the prior-obtained model according to the tracked alterations to provide the modified version of the prior-obtained model; and
correlating the altered patient anatomy as observed from the imaging to the modified version of the prior-obtained model.

19. The computer system of claim 12, wherein the using comprises applying an artificial intelligence (AI) model to identify the at least one object, the AI model configured to identify selected materials based on training the AI model using machine learning and at least one dataset providing reflection or absorption of various wavelengths for varying specific materials.

20. A computer program product comprising:
a computer readable storage medium readable by a processing circuit and storing instructions for execution by the processing circuit for performing a method comprising:
imaging, using at least one spectral imaging camera, an area comprising one or more objects, wherein the imaging comprises obtaining intensity signals for a selective one or more wavelengths or wavelength ranges that correlate to selected material of at least one object of the one or more objects;
using the obtained intensity signals to determine a respective position of each object of the at least one object in space; and
tracking positions of the at one object in space over time.
